(19) United States
(12) Patent Application Publication
(10) Pub. No.: US 2022/0157471 A1
(43) Pub. Date: May 19, 2022

(54) ORGANIZATIONAL GROUP DATA CATEGORIZATION

(71) Applicant: WELLNESS COACHES USA, LLC, Blue Bell, PA (US)

(72) Inventors: Daniel Patterson, Shorewood, MN (US); Mark Walinske, Edina, MN (US); Jason Childs, Rosemount, MN (US); Katherine Rowe, Crystal, MN (US); Michael Larkin, St. Louis, MN (US)

(21) Appl. No.: 17/527,624

(22) Filed: Nov. 16, 2021

Related U.S. Application Data
(60) Provisional application No. 63/114,132, filed on Nov. 16, 2020.

(51) Int. Cl. G16H 50/70; G06Q 30/02; G16H 40/67; G16H 10/60

(52) U.S. Cl. CPC G16H 50/70; G06Q 30/0215; G06Q 40/08; G16H 10/60; G06Q 30/0226; G16H 40/67

(57) ABSTRACT

Disclosed in some examples are systems, methods, and machine-readable media for improved data categorization for group organization. To improve participation, a user may be associated with a group. Anonymized health data from the users may be aggregated to generate one or more target goals, which may be selected by the group as a target goal. A number of groups may compete against other groups, where the competition may be scored based on progress toward their respective selected goals. The generation of target goals and the group progress toward each selected goal may be based on improvement or maximization of user participation.

### PRIORITY

This application claims the benefit of priority to U.S. Provisional Patent Application Ser. No. 63/114,132, filed Nov. 16, 2020, which is incorporated by reference herein in its entirety.

### BACKGROUND

Wearable fitness devices are often used to track daily steps for a user, which may be tracked against a daily goal. However, participation and motivation may be limited to a user's progress against their own daily goal. What is needed is an improved system for tracking and motivating user participation.

### BRIEF DESCRIPTION OF THE DRAWINGS

FIG. 1 shows a first data categorization interface according to some examples of the present disclosure.

FIG. 2 shows a second data categorization interface according to some examples of the present disclosure.

FIG. 3 shows a block diagram of a data categorization method according to some examples of the present disclosure.

FIG. 4 shows a block diagram of a machine within which instructions, for causing the machine to perform any one or more of the methodologies discussed herein, may be executed.

### DETAILED DESCRIPTION

Disclosed in some examples are systems, methods, and machine-readable media for improved data categorization for group organization. To improve participation, a user may be associated with a group. Anonymized health data from the users may be aggregated to generate one or more target goals, which may be selected by the group as a target goal. A number of groups may compete against other groups, where the competition may be scored based on progress toward the team's respective selected goals. The generation of target goals and the group progress toward each selected goal may be based on improvement or maximization of user participation.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

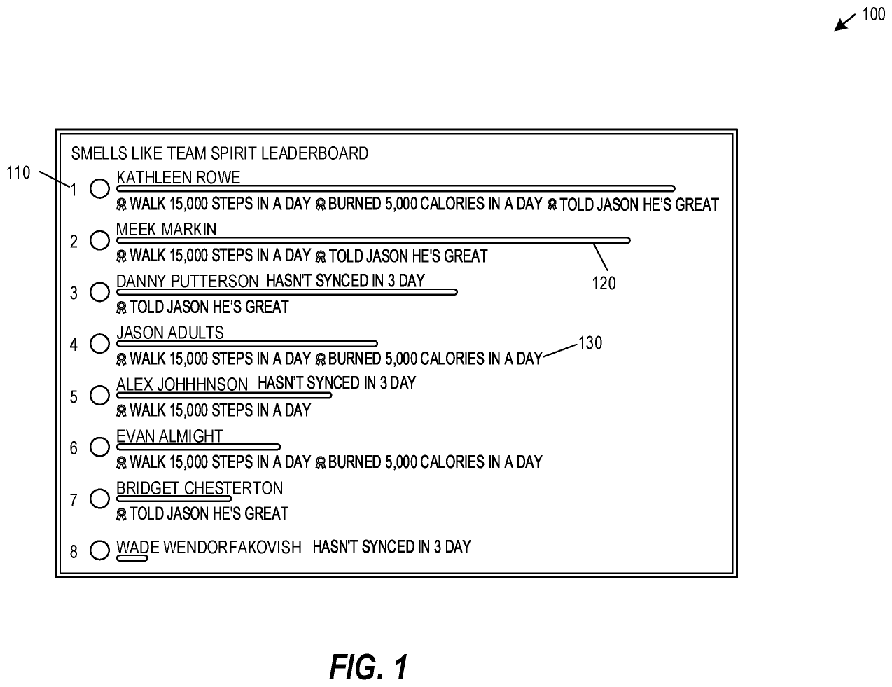

FIG. 1 shows a first data categorization interface 100 according to some examples of the present disclosure. Interface 100 may include a listing of a number of individuals on each team. Each team member may include a member photo (not shown). The member photos may help with understanding who is on each team and ensure the correct individuals are being invited to each team.

Within interface 100, the individuals on each team may be listed according to overall team contributions, where the individual that has contributed most to the team may be listed as the first individual 110. Interface 100 may include a progress bar 120 associated with each individual. Interface 100 may also include individual information 130. The individual information 130 may include calories burned per person, alerts for members that haven't synced, achievements earned for each member, or other individual information. The individual information 130 may be selected to show each achievement, such as to inspire or motivate other team members.

Even though team members are listed separately, the interface 100 and team goals may be organized to encourage team members to work together toward their collective goal. This team collaboration may leverage peer pressure to keep team members motivated, and may improve the resulting team performance.

Each team member may be able to create a team and invite other members to join. Teams may be joined by public groups or by invite only (e.g., anyone may invite or just the team leader). Maximum and minimum team sizes may be restricted to reduce variability within a contest. In an embodiment, the minimum and maximum team size may be based on the types of goals and the ability of a team to reach those goals.

To improve participation and team organization, a team member may only be on one team at a time. Team members may leave a team between contests. If the manager member wants to leave the team, the “manager” role must be passed to another member, or the team would need to be dissolved. Member team history may be preserved.

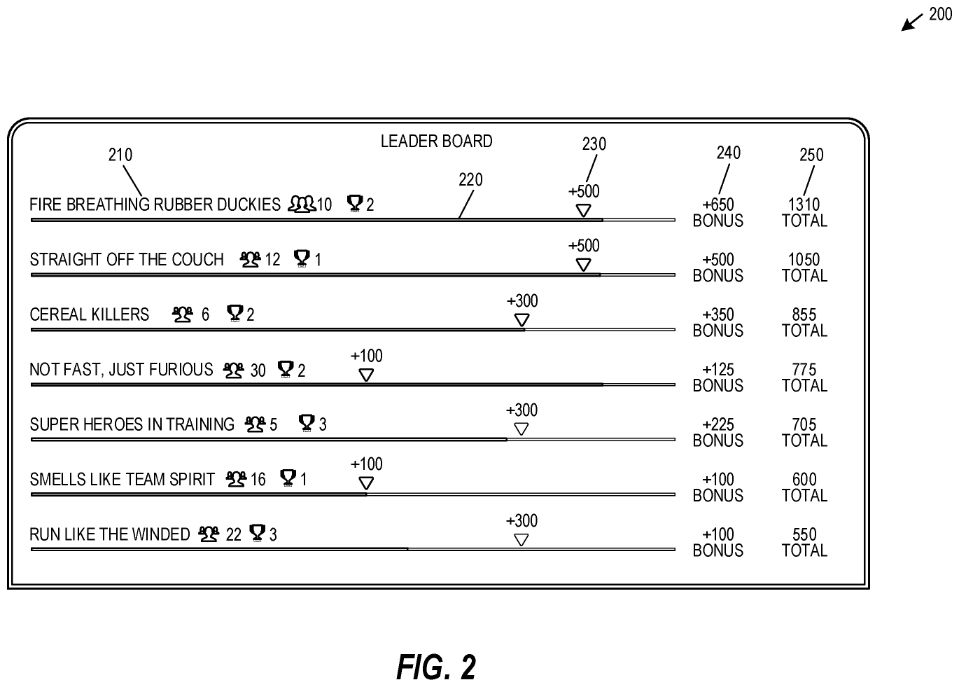

FIG. 2 shows a second data categorization interface 200 according to some examples of the present disclosure. Interface 200 may indicate a leaderboard of a number of teams. Teams may compete against other teams in contests of a predetermined period, such as weekly contest, monthly contest, quarterly contest, or another contest period. In an example, a contest may initially be based on calorie-burning, but may be updated subsequently to use steps, points, or other metrics. All members of each team may earn rewards for a charity or other third-party recipient during a contest. The reward recipient may be switched between contests.

Interface 200 may indicate each team name 210, and may include a number of team members, a number of earned trophies, or other team-specific information. Each team may have an associated progress bar 220, which may indicate progress toward a goal 230. The goal 230 may indicate a location along the progress bar 220 where the team may reach their goal, along with a number of points that may be earned for reaching that goal. Interface 200 may indicate a number of additional bonus points 240, where the bonus points may be based on achieving the goal 230 or other bonus points. Interface 200 may further indicate a team point total 250, which may be used to organize the team listing within interface 200. Team point total 250 may also be used to allocated rewards.

Each team contest goal 230 may be selected prior to or at the beginning of the context. In an example, a number of goal options may be predetermined by a company health care system, and one of those goal options may be selected by voting by the team members. Selection of the goal by team members may improve commitment to achieving the goal by the team members. In an example, a number of goals may be selected or recommended based on a hidden algorithm (e.g., not visible to the team members) that would take into account the previous monthly average of all members, which may be used to generate a number of goal options. For example, goal options may include a Low Goal, a Medium Goal, and a High Goal. In an example, the Low Goal could be 90% of the average, the Medium Goal could be 100% of the average and the High Goal could be 110% of the average.

The reward may be determined based on incremental progress toward that goal. Goals may be based on a conversion (e.g., predetermined ratio) of calories burned to reward dollars. In an example, the Low Goal may have a high floor if the goal is missed and a low ceiling if the goal is achieved. Similarly, the Medium Goal may have a slightly larger range (e.g., lower low, higher high), and the High goal may have the largest range. One example conversion is shown in Table 1:

TABLE 1
Goal Conversion

| | Missed Goal Conversion | Achieve Goal Conversion |
|---|---|---|
| Low Goal | $0.08 per 100 calories | $0.10 per 100 calories |
| Medium Goal | $0.06 per 100 calories | $0.15 per 100 calories |
| High Goal | $0.04 per 100 calories | $0.20 per 100 calories |

The goal conversion may be selected or updated based on budget available for a contest. In an example, if the contest progress indicates that it will reach the goal (e.g., deplete the contest budget) before the projected end of the contest, then the goal conversion may be adjusted periodically. In an example, when there is a limited budget for how much a sponsor may contribute each month, teams may compete for a place each month that earns a portion of the monthly pot.

The amount of the bonus awarded to each team may depend on whether the team selected the Low, Medium or High Goal. Each team may also earn bonuses by reaching individual or team achievements. To reduce the advantage of a large team size, the number of achievements per contest and size of each team may be limited. Total calculations would be made by adding the percent towards the goal plus bonuses. For example, team point total 250 may be calculated as follows:

(% Goal*5)+Goal Bonus+Achievement Bonuses=Team Point Total

Individual points earned may be adjusted based on an adaptive level structure. For example, individuals may receive a point for every calorie burned, and may receive an additional reward for each achieved level. The goal for each next level may increase with each level. For example, level 1 may require 100 points, level 2 may require 110 additional points (e.g., 210 total points), and level 3 may require 120 additional points (e.g., 330 total points.

Rewards may be divided based on the placement of teams at the end of each contest period and based on the size of each team. Each member on the team may earn rewards for their team, which may result in a larger team earning more than a smaller team. To reduce this team size effect, the amount rewarded to each team member may be based on a ranking of all of the teams. An example of such a payout is shown in Table 2:

TABLE 2
Example Team Payout

| Team | Team | Member | Rank | Member | Team | Average |
|---|---|---|---|---|---|---|
| Name | Place | Rank | Cut | Earn | Total | Member Earn |
| Fire | 1 | 1 | 1.96% | $196.08 | $1,873.42 | $187.34 |
| Breathing | | | | | | |
| Rubber | | | | | | |
| Duckies | | | | | | |
| | 1 | 2 | 1.94% | $194.14 | | |
| | 1 | 3 | 1.92% | $192.20 | | |
| | 1 | 4 | 1.90% | $190.25 | | |
| | 1 | 5 | 1.88% | $188.31 | | |
| | 1 | 6 | 1.86% | $186.37 | | |
| | 1 | 7 | 1.84% | $184.43 | | |
| | 1 | 8 | 1.82% | $182.49 | | |
| | 1 | 9 | 1.81% | $180.55 | | |
| | 1 | 10 | 1.79% | $178.61 | | |
| Straight | 2 | 11 | 1.77% | $176.66 | $1,991.85 | $165.99 |
| off the | | | | | | |
| Couch | | | | | | |
| | 2 | 12 | 1.75% | $174.72 | | |
| | 2 | 13 | 1.73% | $172.78 | | |
| | 2 | 14 | 1.71% | $170.84 | | |
| | 2 | 15 | 1.69% | $168.90 | | |
| | 2 | 16 | 1.67% | $166.96 | | |
| | 2 | 17 | 1.65% | $165.02 | | |
| | 2 | 18 | 1.63% | $163.08 | | |
| | 2 | 19 | 1.61% | $161.13 | | |
| | 2 | 20 | 1.59% | $159.19 | | |
| | 2 | 21 | 1.57% | $157.25 | | |
| | 2 | 22 | 1.55% | $155.31 | | |
| Cereal | 3 | 23 | 1.53% | $153.37 | $891.09 | $148.51 |
| Killers | | | | | | |
| | 3 | 24 | 1.51% | $151.43 | | |
| | 3 | 25 | 1.49% | $149.49 | | |
| | 3 | 26 | 1.48% | $147.54 | | |
| | 3 | 27 | 1.46% | $145.60 | | |
| | 3 | 28 | 1.44% | $143.66 | | |

As indicated in Table 2, each team member may use a decreasing percentage. The decreasing percentage may include a linear decreasing percentage, a logarithmic distribution model, a long tail distribution model, or other distribution model. In an example, because a long-tail distribution model may result in a large winning team taking almost all the purse, a logarithmic distribution model may improve the distribution when there is greater variance in team size.

Each team member may have individual achievements. The individual achievements may be included as additional bonuses to improve participation. In an example, individual achievements may be based on engagement (e.g., activity) within a mobile device health application. The individual achievements may start small and grow as each member continues participating in the program. Team achievements may be combined with individual achievements to contribute to a team monthly point total. Individual achievements may also include reaching a daily goal, reaching a daily goal for a predetermined number of consecutive days, reaching 200% of daily goal, inviting a new team member, joining a team, or other achievements.

Within a team, team members may be able to see what achievements other team members have completed. This may further motivate each team member to complete their own individual achievements or to increase participation in the overall team competition. Between teams, members may be able to see what achievements the team has completed to earn their bonus. This may also motivate each team member to complete their own individual achievements or to increase participation in the overall team competition.

Teams may earn money for a selected reward recipient (e.g., charity). Individuals may select a team based on the selected reward recipient. To improve participation, teams may be required to select different reward recipients or different types of reward recipients, such as a variety of local and national charities or other reward recipients. The selection of the reward recipient by the team, and the selection of a team based on the reward recipient may increase participation in the contest. Reward recipients may be changed, such as when the monthly goal is selected at the beginning of each contest cycle.

Reward recipients may include language describing dollar equivalent. For example, $15 buys a backpack, or $8 buys a family a meal. This language describing dollar equivalent will help promote to the team members the effect they will have on the reward recipient. Multiple sponsors may participate within a single group. To clarify the rewards for the teams and team members, one sponsor may be associated with each reward recipient. Outstanding individual contributors could also earn individual contributions to the group recipient or to a separate recipient selected by the user. This may encourage outstanding performers on otherwise low performing teams.

Team members may be segmented to reduce the possible effect of having too many teams potentially depersonalizing the experience. Team members may be segmented by location, department, skill level (e.g., marathoners), or other criteria. This segmentation may facilitate multiple simultaneous contests. Each team member may be limited to see only the contest of their team.

A matching gift may be translated into an achievement bonus for a member's team. In an example, a matching gift receipt may be submitted, and once the gift is approved, a bonus may be applied to the team for that contest.

To facilitate fitness tracking, conventional fitness tracking applications may be used. Fitness applications may include pedometers, wearable fitness trackers, fitness applications (e.g., Google Fit, Apple HealthKit, Fitbit), or other applications. Fitness application data may be normalized or daily limits may be capped, such as for calorie burn calculations. Additional data may be used, such as biometric data, labs data, or other individual information.

Alternatives to fitness tracking may be used. The use of alternate tracking methods may improve uptime and reliability. For example, as an alternative to calorie tracking as a way for people to earn for their team, those that have physical limitations may earn achievements via engagement in a health tracking application.

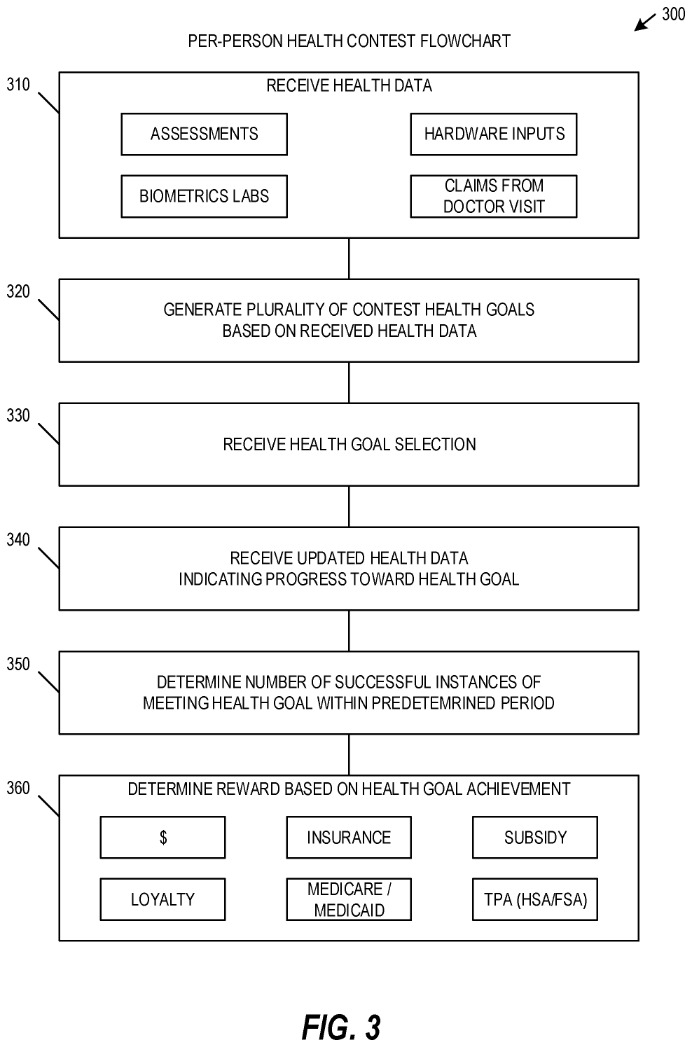

FIG. 3 shows a block diagram of a data categorization method 300 according to some examples of the present disclosure. Method 300 may include receiving 310 a plurality of health data associated with a plurality of individuals. Method 300 may include generating 320 a plurality of health goals based on a normalization of the received plurality of health data. The generation of the plurality of health goals based on the normalization of the received plurality of health data includes generating a health goal that may be predicted to be achievable for each of the plurality of individuals. The plurality of health goals includes various goal levels, such as a low (e.g., conservative) goal, a medium goal, a high (e.g., aggressive) goal or other goals. Each team participating in the competition may receive a number of goals, and each team may select a different goal level.

Method 300 may include receiving 330 a selection of a health goal, the health goal selected from among the generated plurality of health goals. Method 300 may include receiving 340 a plurality of health data from the plurality of individuals, the plurality of health data associated with the health goal. The plurality of health data includes at least one of health assessments, recorded health data from a health tracking device, biometrics labs, or physician data. The plurality of health data may be received from a health sensor associated with each of the plurality of individuals.

Method 300 may include determining 350 a count of a plurality of instances of meeting the health goal. The determination of the count of successful health goal achievement may be based on a predefined period. Method 300 may include determining 360 a reward based on the determined count of the plurality of instances of meeting the health goal.

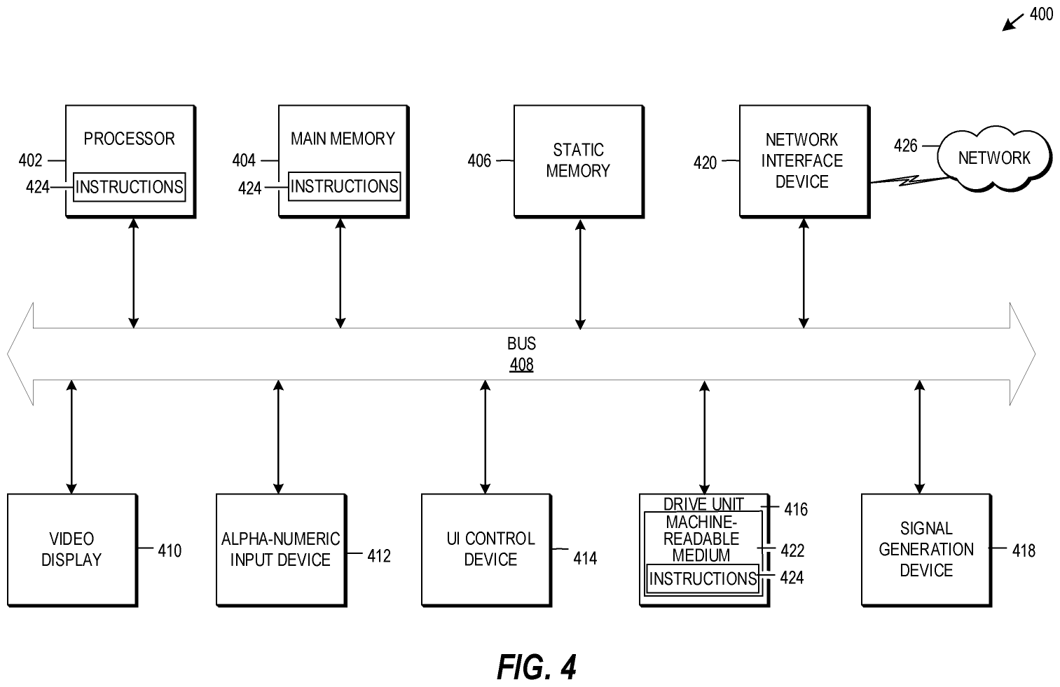

FIG. 4 shows a block diagram of an example computer system 400 within which instructions, for causing the example computer system 400 to perform any one or more of the methodologies discussed herein, may be executed. For example, any one of the components shown in FIG. 1 through FIG. 3 may be or contain one or more of the components described in FIG. 4. In alternative embodiments, the example computer system 400 operates as a standalone device or may be connected (e.g., networked) to other computer systems or machines. In a networked deployment, the example computer system 400 may operate in the capacity of a server or a client machine in server-client network environment, or as a peer machine in a peer-to-peer (or distributed) network environment. The machine may be a personal computer (PC), a notebook PC, a docking station, a wireless access point, a tablet PC, a set-top box (STB), a PDA, a cellular telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. The machine may contain components not shown in FIG. 4 or only a subset of the components shown in FIG. 4.

The example computer system 400 includes a processor 402 (e.g., a central processing unit (CPU), a graphics processing unit (GPU) or both), a main memory 404 and a static memory 406, which communicate with each other via a bus 408. The computer system 400 may further include a video display unit 410 (e.g., a liquid crystal display (LCD) or a cathode ray tube (CRT)). The computer system 400 also includes an alphanumeric input device 412 (e.g., a keyboard), a user interface (UI) navigation device 414 (e.g., a mouse), a disk drive unit 416, a signal generation device 418 (e.g., a speaker) and a network interface device 420.

The disk drive unit 416 includes a machine-readable medium 422 on which is stored one or more sets of instructions and data structures (e.g., software) 424 embodying or used by any one or more of the methodologies or functions described herein. The instructions 424 may also reside, completely or at least partially, within the main memory 404, static memory 406, and/or within the processor 402 during execution thereof by the computer system 400, the main memory 404 and the processor 402 also constituting machine-readable media.

While the machine-readable medium 422 is shown in an example embodiment to be a single medium, the term “machine-readable medium” may include a single medium or multiple media (e.g., a centralized or distributed database, and/or associated caches and servers) that store the one or more instructions or data structures. The term “machine-readable medium” shall also be taken to include any tangible medium that is capable of storing, encoding or carrying instructions for execution by the machine and that cause the machine to perform any one or more of the methodologies of the present invention, or that is capable of storing, encoding or carrying data structures used by or associated with such instructions. The term “machine-readable medium” shall accordingly be taken to include, but not be limited to, solid-state memories, and optical and magnetic media. Specific examples of machine-readable media include non-volatile memory, including by way of example, semiconductor memory devices (e.g., Erasable Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 may further be transmitted or received over a communications network 426 using a transmission medium. The instructions 424 may be transmitted using the network interface device 420 and any one of a number of well-known transfer protocols (e.g., HTTP). Examples of communication networks include a LAN, a WAN, the Internet, mobile telephone networks, Plain Old Telephone (POTS) networks, and wireless data networks (e.g., WiFi and WiMax networks). The term “transmission medium” shall be taken to include any intangible medium that is capable of storing, encoding, or carrying instructions for execution by the machine, and includes digital or analog communications signals or other intangible media to facilitate communication of such software. Network interface device 420 may wirelessly transmit data and may include an antenna.

Certain embodiments are described herein as including logic or a number of components, modules, or mechanisms. Modules may constitute either software modules (e.g., code embodied on a machine-readable medium or in a transmission signal) or hardware modules. A hardware module is a tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired) or temporarily configured (e.g., programmed) to operate in a certain manner and/or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may access the memory device later to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods described herein may be at least partially processor-implemented. For example, at least one or more processors or processor-implemented modules may perform some of the operations of a method. The performance of certain of the operations may be distributed among the one or more processors, not only residing within a single machine, but also deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., APIs).

Example embodiments may be implemented in digital electronic circuitry, in computer hardware, firmware, software, or in combinations of them. Example embodiments may be implemented using a computer program product, for example, a computer program tangibly embodied in an information carrier, for example, in a machine-readable medium for execution by, or to control the operation of, data processing apparatus, for example, a programmable processor, a computer, or multiple computers.

A computer program can be written in any form of programming language, including compiled or interpreted languages, and it can be deployed in any form, including as a stand-alone program or as a module, subroutine, or other unit suitable for use in a computing environment. A computer program can be deployed to be executed on one computer or on multiple computers at one site or distributed across multiple sites and interconnected by a communication network.

In example embodiments, operations may be performed by one or more programmable processors executing a computer program to perform functions by operating on input data and generating output. Method operations can also be performed by, and apparatus of example embodiments may be implemented as, special purpose logic circuitry (e.g., a FPGA or an ASIC).

The computing system can include clients and servers. A client and server are generally remote from each other and typically interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware may be a design choice. FIG. 4 depicts an example hardware (e.g., machine) and software architectures that may be deployed, in various example embodiments.

Example 1 is a system for health data categorization, the system comprising: a memory; and a processor configured to execute instructions to: receive a plurality of health data associated with a plurality of individuals; generate a plurality of health goals based on a normalization of the plurality of health data; receive a selection of a health goal, the health goal selected from among the plurality of health goals; receive a plurality of health data from the plurality of individuals, the plurality of health data associated with the health goal; determine a count of a plurality of instances of meeting the health goal; and determine a reward based on the count of the plurality of instances of meeting the health goal.

In Example 2, the subject matter of Example 1 includes, a plurality of health sensors, wherein: each of the plurality of health sensors is associated with an associated individual within the plurality of individuals; and the plurality of health data is received from the plurality of health sensors.

In Example 3, the subject matter of Examples 1-2 includes, wherein the plurality of health data includes at least one of health assessments, recorded health data from a health tracking device, biometrics labs, or physician Examples data.

In Example 4, the subject matter of Examples 1-3 includes, wherein the generation of the plurality of health goals includes generating a health goal that is predicted to be achievable for each of the plurality of individuals.

In Example 5, the subject matter of Examples 1-4 includes, wherein the plurality of health goals includes at least one of a low goal, a medium goal, and a high goal.

In Example 6, the subject matter of Example 5 includes, wherein: the plurality of individuals forms a first team; and the first team selects a different goal from a second team.

In Example 7, the subject matter of Examples 1-6 includes, wherein the determination of the count of successful health goal achievement is based on a predefined period.

Example 8 is a computer-implemented method for health data categorization, the method comprising: using one or more computer processors to perform operations of: receiving a plurality of health data associated with a plurality of individuals; generating a plurality of health goals based on a normalization of the plurality of health data; receiving a selection of a health goal, the health goal selected from among the plurality of health goals; receiving a plurality of health data from the plurality of individuals, the plurality of health data associated with the health goal; determining a count of a plurality of instances of meeting the health goal; and determining a reward based on the count of the plurality of instances of meeting the health goal.

In Example 9, the subject matter of Example 8 includes, wherein the plurality of health data is received from a health sensor associated with each of the plurality of individuals.

In Example 10, the subject matter of Examples 8-9 includes, wherein the plurality of health data includes at least one of health assessments, recorded health data from a health tracking device, biometrics labs, or physician Examples data.

In Example 11, the subject matter of Examples 8-10 includes, wherein the generation of the plurality of health goals includes generating a health goal that is predicted to be achievable for each of the plurality of individuals.

In Example 12, the subject matter of Examples 8-11 includes, wherein the plurality of health goals includes at least one of a low goal, a medium goal, and a high goal.

In Example 13, the subject matter of Example 12 includes, wherein: the plurality of individuals forms a first team; and the first team selects a different goal from a second team.

In Example 14, the subject matter of Examples 8-13 includes, wherein the determination of the count of successful health goal achievement is based on a predefined period.

Example 15 is a non-transitory machine-readable storage medium, comprising a plurality of instructions that, responsive to being executed with processor circuitry of a computer-controlled circuit, cause the processor circuitry to: receive a plurality of health data associated with a plurality of individuals; generate a plurality of health goals based on a normalization of the plurality of health data; receive a selection of a health goal, the health goal selected from among the plurality of health goals; receive a plurality of health data from the plurality of individuals, the plurality of health data associated with the health goal; determine a count of a plurality of instances of meeting the health goal; and determine a reward based on the count of the plurality of instances of meeting the health goal.

In Example 16, the subject matter of Example 15 includes, wherein the plurality of health data is received from a health sensor associated with each of the plurality of individuals.

In Example 17, the subject matter of Examples 15-16 includes, wherein the plurality of health data includes at least one of health assessments, recorded health data from a health tracking device, biometrics labs, or physician Examples data.

In Example 18, the subject matter of Examples 15-17 includes, wherein the generation of the plurality of health goals includes generating a health goal that is predicted to be achievable for each of the plurality of individuals.

In Example 19, the subject matter of Examples 15-18 includes, wherein the plurality of health goals includes at least one of a low goal, a medium goal, and a high goal.

In Example 20, the subject matter of Example 19 includes, wherein: the plurality of individuals forms a first team; and the first team selects a different goal from a second team.

In Example 21, the subject matter of Examples 15-20 includes, wherein the determination of the count of successful health goal achievement is based on a predefined period.

Example 22 is at least one machine-readable medium including instructions that, when executed by processing circuitry, cause the processing circuitry to perform operations to implement of any of Examples 1-21.

Example 23 is an apparatus comprising means to implement of any of Examples 1-21.

Example 24 is a system to implement of any of Examples 1-21.

Example 25 is a method to implement of any of Examples 1-21.

Although an embodiment has been described with reference to specific example embodiments, it will be evident that various modifications and changes may be made to these embodiments without departing from the broader spirit and scope of the invention. Accordingly, the specification and drawings are to be regarded in an illustrative rather than a restrictive sense. The accompanying drawings that form a part hereof, show by way of illustration, and not of limitation, specific embodiments in which the subject matter may be practiced. The embodiments illustrated are described in sufficient detail to enable those skilled in the art to practice the teachings disclosed herein. Other embodiments may be used and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein, individually or collectively, by the term “invention” merely for convenience and without intending to limit the scope of this application voluntarily to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. A system for health data categorization, the system comprising:
a memory; and
a processor configured to execute instructions to:
receive a plurality of health data associated with a plurality of individuals;
generate a plurality of health goals based on a normalization of the plurality of health data;
receive a selection of a health goal, the health goal selected from among the plurality of health goals;
receive a plurality of health data from the plurality of individuals, the plurality of health data associated with the health goal;
determine a count of a plurality of instances of meeting the health goal; and
determine a reward based on the count of the plurality of instances of meeting the health goal.

2. The system of claim 1, further including a plurality of health sensors, wherein:
each of the plurality of health sensors is associated with an associated individual within the plurality of individuals; and
the plurality of health data is received from the plurality of health sensors.

3. The system of claim 1, wherein the plurality of health data includes at least one of health assessments, recorded health data from a health tracking device, biometrics labs, or physician claims data.

4. The system of claim 1, wherein the generation of the plurality of health goals includes generating a health goal that is predicted to be achievable for each of the plurality of individuals.

5. The system of claim 1, wherein the plurality of health goals includes at least one of a low goal, a medium goal, and a high goal.

6. The system of claim 5, wherein:
the plurality of individuals forms a first team; and
the first team selects a different goal from a second team.

7. The system of claim 1, wherein the determination of the count of successful health goal achievement is based on a predefined period.

8. A computer-implemented method for health data categorization, the method comprising:
using one or more computer processors to perform operations of:
receiving a plurality of health data associated with a plurality of individuals;
generating a plurality of health goals based on a normalization of the plurality of health data;
receiving a selection of a health goal, the health goal selected from among the plurality of health goals;
receiving a plurality of health data from the plurality of individuals, the plurality of health data associated with the health goal;
determining a count of a plurality of instances of meeting the health goal; and
determining a reward based on the count of the plurality of instances of meeting the health goal.

9. The method of claim 8, wherein the plurality of health data is received from a health sensor associated with each of the plurality of individuals.

10. The method of claim 8, wherein the plurality of health data includes at least one of health assessments, recorded health data from a health tracking device, biometrics labs, or physician claims data.

11. The method of claim 8, wherein the generation of the plurality of health goals includes generating a health goal that is predicted to be achievable for each of the plurality of individuals.

12. The method of claim 8, wherein the plurality of health goals includes at least one of a low goal, a medium goal, and a high goal.

13. The method of claim 12, wherein:
the plurality of individuals forms a first team; and
the first team selects a different goal from a second team.

14. The method of claim 8, wherein the determination of the count of successful health goal achievement is based on a predefined period.

15. A non-transitory machine-readable storage medium, comprising a plurality of instructions that, responsive to being executed with processor circuitry of a computer-controlled circuit, cause the processor circuitry to:
receive a plurality of health data associated with a plurality of individuals;
generate a plurality of health goals based on a normalization of the plurality of health data;
receive a selection of a health goal, the health goal selected from among the plurality of health goals;
receive a plurality of health data from the plurality of individuals, the plurality of health data associated with the health goal;
determine a count of a plurality of instances of meeting the health goal; and
determine a reward based on the count of the plurality of instances of meeting the health goal.

16. The non-transitory machine-readable storage medium of claim 15, wherein the plurality of health data is received from a health sensor associated with each of the plurality of individuals.

17. The non-transitory machine-readable storage medium of claim 15, wherein the plurality of health data includes at least one of health assessments, recorded health data from a health tracking device, biometrics labs, or physician claims data.

18. The non-transitory machine-readable storage medium of claim 15, wherein the generation of the plurality of health goals includes generating a health goal that is predicted to be achievable for each of the plurality of individuals.

19. The non-transitory machine-readable storage medium of claim 15, wherein the plurality of health goals includes at least one of a low goal, a medium goal, and a high goal.

20. The non-transitory machine-readable storage medium of claim 19, wherein:
the plurality of individuals forms a first team; and
the first team selects a different goal from a second team.
